(19) United States
(12) Patent Application Publication
(10) Pub. No.: US 2025/0367296 A1
(43) Pub. Date: Dec. 4, 2025

(54) VISCOSITY-REDUCING EXCIPIENT COMPOSITION AND LOW-VISCOSITY HIGHLY CONCENTRATED PROTEIN FORMULATION CONTAINING SAME

(71) Applicant: GREEN CROSS HOLDINGS CORPORATION, Gyeonggi-do (KR)

(72) Inventors: Haeng Eun SONG, Gyeonggi-do (KR); Jae Woon SON, Gyeonggi-do (KR); Jong Moon SON, Gyeonggi-do (KR)

(21) Appl. No.: 18/870,745

(22) PCT Filed: May 22, 2023
(86) PCT No.: PCT/KR2023/006891

§ 371 Date: Dec. 2, 2024

(30) Foreign Application Priority Data
Jun. 3, 2022 (KR) 10-2022-0068278

(51) Int. Cl. A61K 47/20; A61K 9/00; A61K 47/18; A61K 47/22; C07K 16/06

(52) U.S. Cl. CPC A61K 47/20; A61K 9/0019; A61K 47/183; A61K 47/22; C07K 16/06

(57) ABSTRACT

Disclosed are an excipient composition for reducing the viscosity of protein pharmaceuticals and a low-viscosity, high-concentration protein composition formulation containing the same which have a remarkably low viscosity at a high protein concentration of about 20% or more, are convenient to manufacture, store, and use, and are particularly useful for the preparation of injections for antibody treatment for subcutaneous administration and intramuscular administration. In particular, the high-concentration protein formulation can be advantageously used immediately after storage without separate preheating because it maintains low viscosity even when stored at low temperature.

### BACKGROUND OF THE INVENTION

### Field of the Invention

The present invention relates to an excipient composition for reducing the viscosity of protein pharmaceuticals and a low-viscosity, high-concentration protein composition formulation containing the same.

### Description of the Related Art

Monoclonal antibodies (mAbs) are in the limelight as therapeutic agents targeting various types of diseases and therapeutic antibodies for a variety of diseases including cancer, neurodegenerative diseases, and immune diseases are clinically approved and then used and are continuously developed (Nature Reviews Immunology 6(5):343-357).

Conventional antibody pharmaceuticals are generally administered via intravenous injection and are formulated into low-concentration IV formulations of less than about 10 mg/mL for administration of typical effective doses of about 100 mg to about 250 mg. However, the low-concentration IV formulations have disadvantages in that administration to patients takes a lot of time and always requires a visit to hospitals (Canadian Oncology Nursing Journal 25(3):341-346.).

Recently, in order to overcome this disadvantage of 5 low-concentration IV antibody formulations, efforts to develop subcutaneous injection (SC) formulations of antibody drugs have been actively made. In general, subcutaneous formulations have advantages of a small volume of 1 to 1.5 mL, rapid administration and, particularly, self-injection (J Pharm Sci 93(6): 1390-1402; Pharm Res. 2013, 30, 7). The 10 effective dose of the antibody as an active ingredient is about 100 to about 250 and mg, a formulation for subcutaneous administration of 1 to 1.5 mg/ml has a high concentration of about 100 mg/ml to about 300 mg/ml.

However, disadvantageously, these high-concentration antibody formulations induce protein-protein interactions between antibodies, form reversible or irreversible aggregates, induce immunogenicity, and exhibit high viscosity beyond a pharmaceutically acceptable level (about 20 cP) (Industrial & Engineering Chemistry Research, 55(43), 11225-11234). In addition, high-viscosity formulations may cause difficulties in the protein preparation process, especially in filtration.

The high viscosity of high-concentration antibody formulations is reported to be due to various protein-protein interactions as the distance between antibodies becomes closer (Mol Pharmaceutics 12(1): 127-139.), and a method of manipulating the sequence of amino acids that trigger interactions between antibodies to reduce the viscosity of antibody formulations has been reported (J. Pharm. Sci. 2013, 102, 8). However, this method has problems in that it is specific to individual antibodies, takes a lot of time to develop, and has a limitation of universal inapplicability due to the common issue of reduced viscosity that occurs not only in antibodies but also in various protein pharmaceuticals other than antibodies.

Therefore, research to reduce the viscosity of protein pharmaceuticals using various excipients has been conducted. A method of inhibiting the interaction between proteins by adding salts such as representative viscosity-reducing excipients, NaCl (Mol Pharmaceutics 12(1):127-139), calcium chloride or magnesium chloride (U.S. Pat. No. 7,758,860), arginine hydrochloride, sodium thiocyanate, ammonium thiocyanate, ammonium sulfate, ammonium chloride, calcium chloride, zinc chloride, or sodium acetate (U.S. Pat. No. 7,666,413) has been reported. However, recent reports have shown that the addition of salt may have no effect on the viscosity depending on the protein (Biophys J 106(8):1763-1770; Pharm Res 29(11):3102-3109), or rather may cause an increase in viscosity (Soft Matter 10(6):894-902).

The use of charged amino acids as viscosity-reducing excipients in protein formulations has also been reported. In particular, arginine has been reported to reduce protein aggregation and viscosity (Biochemistry 44(12):4919-4925). In addition, it has been reported that amino acids such as histidine and lysine decrease the viscosity of protein formulations (Pharmaceutical Research, vol. 29, no. 11, Jun. 13, 2012, p. 3182-3189).

Benzyl benzoate (Miller et al, Langmuir 26:1067-1074, 2010), benzyl acetate, ethanol, methyl ethyl ketone (Srinivasan et al, Pharm. Res. 30: 1749-1757) and the like have been reported as viscosity-reducing excipients using an organic compound. However, none thereof had a sufficient viscosity-reducing effect at a level (about 20 cP or less) appropriate for administration such as subcutaneous injection.

FDA-approved antibody pharmaceuticals for administration such as subcutaneous injection include Cuvitru, Hizentra, and Xembify, which contain 20% IgG antibody. Cuvitru and Xembify use glycine (Cuvitru (250 mM) and Xembify (160-260 mM)) as stabilizer, and Hizentra uses proline (250 mM) as a stabilizer. These pharmaceuticals should be refrigerated, but still exhibit a high viscosity when refrigerated, which is inconvenient for use.

Under this background, the present inventors have made diligent efforts to develop viscosity-reducing formulations for high-concentration protein pharmaceuticals. As a result, the present inventors found that benzenesulfonic acid and camphorsulfonic acid are added to a conventional protein therapeutic composition containing proline or glycine, the viscosity greatly decreased and completed the present invention, based thereon.

### SUMMARY OF THE INVENTION

It is one object of the present invention to provide a viscosity-reducing excipient composition and a low-viscosity, high-concentration protein formulation containing the same.

It is another object of the present invention to provide a method for reducing the viscosity of a liquid composition containing protein.

In accordance with one aspect of the present invention, the above and other objects can be accomplished by the provision of a pharmaceutical composition containing at least one protein; glycine or proline; benzenesulfonic acid; and camphorsulfonic acid.

In accordance with another aspect of the present invention, provided is a viscosity-reducing excipient composition containing benzenesulfonic acid and camphorsulfonic acid, or a pharmaceutically acceptable salt thereof.

In accordance with another aspect of the present invention, provided is a method of reducing the viscosity of a composition containing protein comprising adding a benzenesulfonic acid and camphorsulfonic acid as a viscosity-reducing excipient to a composition containing at least one protein, and glycine or proline.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the viscosity depending on the concentration between a 20% IgG base formulation containing proline and a formulation prepared by adding benzenesulfonic acid and/or camphorsulfonic acid to the 20% IgG base formulation containing proline.

FIG. 2 is a graph showing viscosity depending on concentration between a 20% IgG base formulation containing glycine and a formulation prepared by adding benzenesulfonic acid and/or camphorsulfonic acid to 20% IgG base formulation containing glycine.

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

Protein therapeutics are target-specific therapeutics for specific diseases and therapeutic proteins for various diseases such as cancer and immune-related diseases are developed and used. Currently, protein therapeutics are mostly administered as low-concentration formulations by intravenous injection, but development of high-concentration formulations for effective subcutaneous or intramuscular administration has recently been actively conducted. However, high-concentration protein compositions have a high viscosity due to protein interaction, causing difficulties in manufacture, management, and administration, and discomfort such as pain to patients.

Various excipients and combinations for use in various low-viscosity, highly-concentrated protein formulations have been reported to date and actually exhibited a slight reduction in viscosity. There are only a few excipient compositions that exhibit a viscosity-reducing effect sufficient for clinical use. In particular, previously reported viscosity-reducing excipients exhibit an increase in the viscosity of the formulation when refrigerated at low temperatures, which is essential for the preservation of protein pharmaceuticals. Therefore, preheating is essential before use. During this process, disadvantageously, viscosity-reducing excipients may be deformed or contaminated.

In one embodiment of the present invention, it was found that, when a combination of benzenesulfonic acid and camphorsulfonic acid was added to a conventional protein therapeutic composition containing proline or glycine, the viscosity of the protein formulation was significantly reduced, and in particular, was sufficiently low for immediate administration even under low-temperature refrigeration conditions.

In one aspect, the present invention provides a pharmaceutical composition containing at least one protein; glycine or proline; benzenesulfonic acid; and camphorsulfonic acid.

As used herein, the term “protein” refers to an amino acid polymer forming a polypeptide having a sufficient length to produce a tertiary structure linked by a peptide bond. In general, proteins are classified into high-molecular-weight proteins of 100 kDa or more and low-molecular-weight proteins of less than 100 kDa. In some embodiments of the present invention, the protein may have at least one biological effect, and preferably, the protein may be a “therapeutic protein” having a prophylactic, ameliorative, or therapeutic effect for at least one disease.

Preferably, the protein may be an antigen-binding protein capable of specifically binding to a target substance or antigen.

As used herein, the term “antigen-binding protein” refers to a protein capable of recognizing an antigen and specifically binding thereto. In a broad sense, non-limiting examples of the antigen-binding protein include antibodies or antigen-binding fragments thereof, antibody-like proteins (ALP) such diabodies, as pentambodies, repebodies, or affimers, and antigen-binding peptides such as C7 peptides.

In some embodiments of the present invention, the protein is preferably an antibody or binding-fragment thereof.

The term “antibody” means a protein capable of neutralizing, blocking, inhibiting, eliminating, reducing or interfering with a biological activity.

The antibody of the present invention includes, but is not limited to, monoclonal antibodies, multispecific antibodies, human antibodies, humanized antibodies, chimeric antibodies, single chain FVs (scFVs), single-chain antibodies, Fab fragments, F(ab′) fragments, disulfide-bond Fvs (sdFVs), anti-idiotypic (anti-Id) antibodies, epitope-binding fragments of such antibodies, and the like.

The term “monoclonal antibody” refers to an identical antibody, which is obtained from a population of substantially homogeneous antibodies, that is, each antibody constituting the population, excluding possible naturally occurring mutations that may be comprised in trivial amounts. Monoclonal antibodies are highly specific and are thus induced against a single antigenic site.

The term “epitope” refers to a protein determinant to which an antibody can specifically bind. Epitopes usually consist of a group of chemically active surface molecules, such as amino acid or sugar side chains, and generally have not only specific three-dimensional structural characteristics but also specific charge characteristics. Three-dimensional epitopes are distinguished from non-three-dimensional epitopes in that a bond to the former is broken in the presence of a denatured solvent, while a bond to the latter is not broken.

The whole antibody has a structure having two full-length light-chains and two full-length heavy-chains, and each light-chain is bonded to the heavy-chain by a disulfide bond. The whole antibody includes IgA, IgD, IgE, IgM, and IgG, and IgG may include IgG1, IgG2, IgG3, and IgG4 subtypes.

The heavy-chain constant region has gamma (γ), mu (μ), alpha (α), delta (δ) and epsilon (ε) types, and is subclassified into gamma 1 (γ1), gamma 2 (γ2), gamma 3 (γ3), gamma 4 (γ4), alpha 1 (γ1), and alpha 2 (α2). The light-chain constant region has kappa (κ) and lambda (λ) types.

The term “antigen-binding fragment of an antibody” or “antibody fragment” refers to a fragment that has antigen-binding function and includes Fab, F(ab′), F(ab′)2, Fv and the like. Among the antibody fragments, Fab refers to a structure including a variable region of each of the heavy-chain and the light-chain, the constant region of the light-chain, and the first constant domain (CH1) of the heavy-chain, each having one antigen-binding site. Fab′ is different from Fab in that it further includes a hinge region including at least one cysteine residue at the C-terminus of the CH1 domain of the heavy-chain. F(ab′)2 is created by a disulfide bond between cysteine residues in the hinge region of Fab′. Fv is the minimal antibody fragment having only a heavy-chain variable region and a light-chain variable region. Two-chain Fv is a fragment in which the variable region of the heavy-chain and the variable region of the light-chain are linked by a non-covalent bond, and single-chain Fv (scFv) is a fragment in which the variable region of the heavy-chain and the variable region of the light-chain are generally linked by a covalent bond via a peptide linker therebetween, or are directly linked at the C-terminal, forming a dimer-shaped structure, like the two-chain Fv. Such antibody fragments may be obtained using proteases (e.g., Fab can be obtained by restriction-cleaving the complete antibody with papain, and the F(ab′)2 fragment can be obtained by restriction-cleaving the complete antibody with pepsin), and may be produced using genetic recombination techniques.

As used herein, the term “Fv fragment” is an antibody fragment containing antibody complete recognition and binding sites. Such a region includes a dimer that consists of one heavy-chain variable domain and one light-chain variable domain substantially tightly covalently linked to each other, for example, through scFv.

As used herein, the term “Fab” fragment contains a variable domain and a constant domain of the light-chain and a variable domain and a first constant domain (CH1) of the heavy-chain. A F(ab′)₂ antibody fragment generally includes a pair of Fab fragments covalently linked near the carboxyl terminal thereof via a hinge cysteine therebetween.

As used herein, the “single chain Fv” or “scFv” antibody fragment includes VH and VL domains of the antibody, wherein these domains are comprised in a single polypeptide chain. The Fv polypeptide may further include a polypeptide linker between the VH domain and the VL domain in order for the scFv to form a target structure for antigen binding.

For example, in some embodiments of the present invention, the antibody is in an Fv form (for example, scFv) or a complete antibody form. In addition, the heavy-chain constant region may be selected from gamma (γ), mu (u), alpha (α), delta (δ) and epsilon (c) isotypes. For example, the constant region may be gamma 1 (IgG1), gamma 3 (IgG3) or gamma 4 (IgG4). The light-chain constant region may be kappa or lambda.

As used herein, the term “heavy chain” encompasses both a full-length heavy chain, which includes a variable domain (VH), containing an amino acid sequence having a variable region sequence sufficient for imparting specificity to an antigen and three constant domains (CH1, CH2 and CH3), and a fragment thereof. As used herein, the term “light chain” encompasses both a full-length light chain, which includes a variable domain (VL) containing an amino acid sequence having a variable region sequence sufficient for imparting specificity to an antigen and a constant domain (CL), and a fragment thereof.

A part of the heavy chain and/or light chain is identical to or homologous with the corresponding sequence in an antibody derived from a particular species or belonging to a particular antibody class or subclass, while the remaining chain(s) include “chimeric” antibodies (immunoglobulins) which are identical to or homologous with corresponding sequences in an antibody derived from another species or belonging to another antibody class or subclass, as well as fragments of such antibody exhibiting the desired biological activity.

As used herein, the term “antibody variable domain” refers to the light- and heavy-chain regions of an antibody molecule including the amino acid sequences of a complementarity-determining region (CDR; i.e., CDR1, CDR2, and CDR3) and a framework region (FR). VH refers to a variable domain of the heavy chain. VL refers to a variable domain of the light chain.

The term “complementarity-determining region” (CDR, that is, CDR1, CDR2, and CDR3), refers to an amino acid residue of the antibody variable domain, which is necessary for antigen binding. Each variable domain typically has three CDR regions, identified as CDR1, CDR2, and CDR3.

The non-human (e.g., murine) antibody of the “humanized” form is a chimeric antibody containing a minimal sequence derived from non-human immunoglobulin. The humanized antibody is a human immunoglobulin (receptor antibody) in which a residue from the hypervariable region of a receptor is replaced with a residue from the hypervariable region of a non-human species (donor antibody) such as a mouse, rat, rabbit or non-human primate having the desired specificity, affinity and ability.

In some embodiments of the present invention, the protein may be an antibody, preferably a monoclonal antibody, more preferably an IgG type antibody. In some embodiments of the present invention, the antibody may be a variety of therapeutic antibodies that are commercially available or under research and development in the art.

In some embodiments of the present invention, the protein may be a therapeutic protein other than an antibody. For example, the protein may be an enzyme, fusion protein, PEGylated protein, vaccine, biologically active protein, or protein mixture.

As used herein, the term “enzyme” refers to a protein or functional fragment thereof that catalyzes the biochemical conversion of a molecule into a desired product.

As used herein, the term “fusion protein” refers to a protein produced from two different genes encoding two separate proteins. The fusion protein is generally produced through recombinant DNA techniques known to those skilled in the art. Two proteins (or protein fragments) exhibit characteristics derived from both parental proteins by which they are covalently fused together.

In some embodiments of the present invention, the protein may be a conjugate with another compound. For example, the conjugate may be an antibody-drug conjugate, but is not limited thereto.

In some embodiments of the present invention, the protein may be contained as an “active ingredient” having a medicinally and/or pharmacologically active effect.

In some embodiments of the present invention, the protein may be comprised in a pharmaceutically active amount. For example, a pharmaceutically active single dose of the antibody therapeutic agent may be about 150 mg to about 250 mg, but is not limited thereto.

The pharmaceutical composition of the present invention may contain a high concentration of protein.

In some embodiments of the present invention, the concentration of the protein is about 1 mg/mL or more, about 10 mg/mL or more, or about 50 mg/mL or more, more preferably about 150 mg/mL or more, more preferably about 200 mg/mL or more.

In some embodiments of the present invention, the concentration of the protein is about 10 mg/mL to about 5,000 mg/mL, preferably about 50 mg/mL to about 2,500 mg/mL, more preferably about 100 mg/mL to about 1,000 mg/mL, more preferably about 100 mg/mL to about 500 mg/mL, most preferably about 150 mg/mL to about 500 mg/mL, but is not limited thereto.

In some embodiments of the present invention, the concentration of the protein is about 1 w/v % to about 70 w/v %, more preferably about 5 w/v % to about 50 w/v %, more preferably about 7 w/v % to about 35 w/v %, more preferably about 10 w/v % to about 25 w/v %, but is not limited thereto. In one embodiment of the present invention, the protein is comprised at a concentration of about 200 mg/mL (about 20% w/v), but is not limited thereto.

In some embodiments of the present invention, the glycine or proline, benzenesulfonic acid and camphorsulfonic acid may be comprised as excipients.

As used herein, the term “excipient” refers to additives other than active ingredients having one or more physical/chemical effects.

In some embodiments of the present invention, the glycine or proline may be comprised as a stabilizer. As used herein, the term “stabilizer” refers to an additive used to impart physical stability or chemical stability to the pharmaceutical composition. For example, at least about 95% of the bioactive protein molecules may retain the bioactivity of the formulation after storage at 4° C. for 24 months, or under equivalent solution conditions at elevated temperatures, such as storage at 40° C. for 1 month, but is not limited thereto.

In some embodiments of the present invention, the glycine or proline may affect one or more physical and/or chemical properties such as viscosity-reducing effects and isotonicity of the composition, in addition to the stabilizing effect of protein activity.

In some embodiments of the present invention, the glycine or proline may be comprised at a concentration of about 10 mM to about 1,000 mM, preferably about 50 mM to about 500 mM, more preferably about 100 mM to about 400 mM, more preferably about 150 mM to about 300 mM. In one embodiment of the present invention, the composition containing 250 mM glycine or proline was prepared, but is not limited thereto.

In one embodiment of the present invention, it was found that, when benzenesulfonic acid and camphorsulfonic acid were further administered, the viscosity of the composition was remarkably reduced, and in particular, the composition had low viscosity even under a low-temperature refrigeration condition of 4° C.

As used herein, the term “benzenesulfonic acid” is an aromatic sulfonic acid and has a structure represented by the following Formula I.

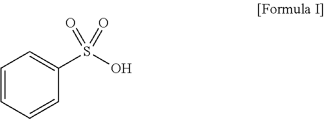

The benzenesulfonic acid may be produced through various conventionally known methods and is typically produced by sulfonating benzene using sulfuric acid (Otto Lindner, Lars Rodefeld (2005). “Benzenesulfonic Acids and Their Derivatives”. Ullmann's Encyclopedia of Industrial Chemistry.), but is not limited thereto.

In some embodiments of the present invention, the benzenesulfonic acid may be comprised as benzenesulfonate, which is a pharmaceutically acceptable salt thereof. The benzenesulfonic acid may be comprised as a salt of alkali metal or alkaline earth metal such as sodium, potassium, lithium, calcium, or magnesium, but is not limited thereto.

In some embodiments of the present invention, the benzenesulfonic acid (BA) is comprised at a concentration of about 5 mg/mL to about 30 mg/mL, preferably about 10 mg/mL to about 27.5 mg/mL, more preferably about 10 mg/mL to about 25 mg/mL, most preferably about 10 mg/mL to about 20 mg/mL.

As used herein, the term “camphorsulfonic acid (CA)” is an aromatic sulfonic acid, is expressed as 1S-(+)-10-camphorsulfonic acid, and has a structure represented by the following Formula II.

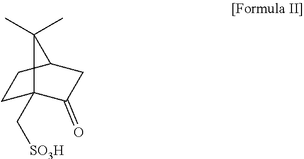

The camphorsulfonic acid can be prepared through various methods known in the art and is typically prepared by sulfonation of a methyl group, preferably, formation of alkene from a methyl group, followed by sulfonation of alkene and rearrangement of semipinacol (Bruckner, Reinhard (2002). Advanced organic chemistry: reaction mechanisms.), but is not limited thereto.

In some embodiments of the present invention, the camphorsulfonic acid may be comprised as camphorsulfonate, which is a pharmaceutically acceptable salt thereof. The camphorsulfonic acid may be comprised as a salt of alkali metal or alkaline earth metal such as sodium, potassium, lithium, calcium, or magnesium, but is not limited thereto.

In some embodiments of the present invention, the camphorsulfonic acid (CA) is comprised at a concentration of about 5 mg/mL to about 30 mg/mL, preferably about 10 mg/mL to about 27.5 mg/mL, more preferably about 10 mg/mL to about 25 mg/mL, most preferably about 10 mg/mL to about 20 mg/mL.

In the examples of the present invention, it was found that the combination of benzenesulfonic acid and camphorsulfonic acid exhibits a significant viscosity reduction effect, based on the same total concentration, compared to benzenesulfonic acid or camphorsulfonic acid alone.

In some embodiments of the present invention, the total concentration of benzenesulfonic acid and camphorsulfonic acid is about 10 mg/mL to about 60 mg/mL, preferably about 20 mg/mL to about 50 mg/mL, more preferably about 20 mg/mL to about 40 mg/mL, most preferably about 30 mg/mL to 40 mg/mL.

In some embodiments of the present invention, the benzenesulfonic acid and the camphorsulfonic acid may be comprised in a weight ratio of 1:0.5 to 1:2.5, preferably in a weight ratio of 1:1 to 1:2.5, more preferably in a weight ratio of 1:2.0, 1:1 or 1:0.75.

In some embodiments of the present invention, the benzenesulfonic acid and the camphorsulfonic acid may be comprised as viscosity-reducing excipients.

As used herein, the term “viscosity-reducing excipient” or “viscosity-reducing agent” refers to a compound that acts to reduce the viscosity of a solution containing the viscosity-reducing agent, compared to a solution not containing the viscosity-reducing agent.

As used herein, the term “viscosity” means the resistance to the flow of a fluid (liquid or gas) or movement of neighboring parts relative to each other. The reciprocal of viscosity can be expressed as fluidity. The viscosity may be related to the concept of shear force and can be understood as the effect of different layers of a fluid that exert a shear force on one another, or against another surface as they move relative to each other. The viscosity can be expressed as Ns/m², also known as Pascal*second (Pa*s). The viscosity may be “kinematic viscosity” or “absolute viscosity”. “Viscosity” used herein is generally understood as “absolute viscosity”.

The kinematic viscosity is a measure of the rate at which momentum is transferred through a fluid. Kinematic viscosity is measured in Stokes (St). Kinematic viscosity may be measured through the resistive flow of a fluid under the influence of gravity. When two fluids having an identical volume and different viscosities are injected into the same capillary viscometer and allowed to flow by gravity, the fluid with a higher viscosity takes longer to flow through the capillary than the fluid having a lower viscosity. For example, if one fluid takes 200 seconds(s) to complete its flow and the other fluid takes 400 seconds(s) to complete its flow, the second fluid can be expressed as twice as viscous as the first on a kinematic viscosity basis. The unit of kinematic viscosity can be expressed as length²/hour. Kinematic viscosity often expressed in centistokes (cSt), and the SI unit for kinematic viscosity is cm²/s, which equals 1St.

As used herein, the term “absolute viscosity” may be used interchangeably with “dynamic viscosity” or “simple viscosity” and means the product of kinematic viscosity and fluid density. Absolute viscosity is expressed in centipoise (cP). The SI unit for absolute viscosity is the milliPascal-second (mPa-s), where 1 cP=1 mPa*s.

In some embodiments of the present invention, the viscosity can be measured through various methods known in the art, viscometers and rheometers. For example, the viscosity may be measured using a glass capillary viscometer, a Stormer viscometer, a vibration viscometer, or the like, but is not limited thereto.

In some embodiments of the present invention, the pharmaceutical composition may have low viscosity. In some embodiments of the present invention, the pharmaceutical composition may have a viscosity of 50 cP or less, preferably about 40 cP or less, about 35 cP or less, or about 30 cP or less, more preferably about 20 cP or less or about 15 cP or less, but is not limited thereto.

In some embodiments of the present invention, the pharmaceutical composition has a viscosity reduced by at least 10%, at least 20%, at least 30%, at least 40%, or at least 50%, compared to a formulation not containing benzenesulfonic acid and camphorsulfonic acid as viscosity-reducing excipients.

In some embodiments of the present invention, the viscosity of the pharmaceutical composition may vary depending on temperature. In one embodiment of the present invention, it was found that when benzenesulfonic acid and camphorsulfonic acid were contained as viscosity reducing excipients, a great decrease in viscosity was obtained at about 4° C., which is a storage temperature of general protein formulations.

In some embodiments of the present invention, the pharmaceutical composition may have a viscosity of 50 cP or less at about 4° C., preferably about 40 cP or less at about 4° C., about 35 cP or less at about 4° C., or about 30 cP or less at about 4° C., more preferably about 20 cP or less at about 4° C., or about 15 cP or less at about 4° C., but is not limited thereto.

In some embodiments of the present invention, the pharmaceutical composition has a viscosity of 50 cP or less, preferably about 40 cP or less, about 35 cP or less, or about 30 cP or less, more preferably about 20 cP or less or about 15 cP or less, even when stored at about 4° C. for at least 4 hours, at least 6 hours, at least 8 hours, at least 12 hours, or at least 1 day.

The pharmaceutical composition of the present invention can be useful to prepare a high-concentration protein formulation.

The pharmaceutical composition of the present invention may further contain a suitable carrier, excipient and diluent commonly used in pharmaceutical compositions, in addition to the proline or glycine, and benzenesulfonic acid and camphorsulfonic acid.

Examples of the carrier, excipient or diluent that may be contained in the composition include lactose, dextrose, sucrose, sorbitol, mannitol, xylitol, erythritol, maltitol, starch, acacia rubber, alginate, gelatin, calcium phosphate, calcium silicate, cellulose, methyl cellulose, water, microcrystalline cellulose, polyvinyl pyrrolidone, methyl hydroxybenzoate, propyl hydroxybenzoate, talc, magnesium stearate, mineral oil, and the like. The formulation of the composition may be prepared using a commonly used diluent or excipient such as a filler, extender, binder, wetting agent, disintegrant, or surfactant.

In particular, pharmaceutical excipients useful for liquid protein formulations are well known to those skilled in the art. Non-limiting examples thereof include: liquid solvents or co-solvents; sugars or sugar alcohols such as mannitol, trehalose, sucrose, sorbitol, fructose, maltose, lactose or dextran; surfactants such as TWEEN® 20, 60, or 80 (polysorbate 20, 60, or 80); buffer; preservatives such as benzalkonium chloride, benzethonium chloride, tertiary ammonium salts, and chlorhexidine diacetate; carriers such as poly (ethylene glycol) (PEG); antioxidants such as ascorbic acid, sodium metabisulfite, and methionine; chelating agents such as EDTA or citric acid; or biodegradable polymers such as water-soluble polyesters; cryoprotectants; lyophilization protectants; bulking agents; and stabilizers. Further, other pharmaceutically acceptable carriers, excipients or stabilizers described in Remington: “The Science and Practice of Pharmacy” 20^{th} edition, Alfonso R Gennaro, Ed., Lippincott Williams & Wilkins (2000) may be contained in the protein formulation described herein as long as they do not adversely affect the desired characteristics of the formulation.

The pharmaceutical composition according to the present invention can be prepared into a suitable formulation using a pharmaceutically inactive organic or inorganic carrier. That is, when the formulation is a tablet, a coated tablet, a dragée or a hard capsule, it may contain lactose, sucrose, starch or a derivative thereof, talc, calcium carbonate, gelatin, stearic acid, or a salt thereof. In addition, when the formulation is a soft capsule, it may contain a vegetable oil, wax, fat, or semi-solid or liquid polyol. In addition, when the formulation is in the form of a solution or syrup, it may contain water, polyol, glycerol, vegetable oil, or the like.

The pharmaceutical composition according to the present invention may further contain a preservative, a stabilizer, a wetting agent, an emulsifier, a solubilizing agent, a flavoring agent, a colorant, an osmotic pressure regulator, an antioxidant or the like, in addition to the carrier.

The pharmaceutical composition according to the present invention may be administered in a pharmaceutically effective amount, and the term “pharmaceutically effective amount” refers to an amount sufficient to treat a disease at a reasonable benefit/risk ratio applicable to all medical treatments, and the effective dosage level may be determined depending on a variety of factors including the type of the disease of the patient, the severity of the disease, the activity of the drug, the sensitivity of the patient to the drug, the administration time, the administration route, the excretion rate, the treatment period, drugs used concurrently therewith, and other factors well-known in the pharmaceutical field. The pharmaceutical composition of the present invention may be administered as a single therapeutic agent or in combination with other therapeutic agents, may be administered sequentially or simultaneously with a conventional therapeutic agent, and may be administered in one or multiple doses. Taking into consideration these factors, it is important to administer the minimum amount sufficient to achieve maximum efficacy without side effects, and the amount can be easily determined by those skilled in the art.

In some embodiments of the present invention, the pharmaceutical composition may be administered through various routes. For example, the pharmaceutical composition may be administered to the patient by subcutaneous, intra-muscular, intra-peritoneal, intra-cerebrospinal, intra-venous, intra-articular, intra-synovial, intra-thecal, oral, or topical administration, or by inhalation, most preferably subcutaneous or intramuscular administration, but is not limited thereto. When the pharmaceutical composition of the present invention is subcutaneously administered, an injection device (e.g., INJECT-EASE™ and GENJECT™), a syringe pen (e.g., GENPEN™), a needleless device (e.g., MEDIJECTOR™ and BIOJECTOR™), or a subcutaneous patch delivery system may be used.

The pharmaceutical formulation according to the present invention may be provided in a form stored in a glass ampoule or plastic container for administration, or a ready-to-inject form such as a pre-filled syringe. In particular, when the formulation is administered by subcutaneous injection or intramuscular injection, it may be subdivided into a volume of about 1 mL to 10 mL, but is not limited thereto, and if necessary, a dilution using an appropriate solution such as physiological saline or glucose injection may be administered.

The pharmaceutical composition of the present invention exhibits high stability and low viscosity of an immediately administrable level even at a low temperature of 4° C., so that it can be immediately administered even after refrigerated storage.

The pharmaceutical composition of the present invention can be administered immediately without preheating after separate refrigerated storage.

The pharmaceutical composition of the present invention can be administered immediately without a pre-administration process such as pre-heating or dilution, thus preventing contamination or modification and/or inactivation of protein (preferably as an active ingredient) that may occur during the pre-administration process.

In another aspect, the present invention relates to an excipient for reducing viscosity containing benzenesulfonic acid and camphorsulfonic acid.

In some embodiments of the present invention, the benzenesulfonic acid may be comprised as benzenesulfonate, which is a pharmaceutically acceptable salt thereof. The benzenesulfonic acid may be comprised as a salt of alkali metal or alkaline earth metal such as sodium, potassium, lithium, calcium, or magnesium, but is not limited thereto.

In some embodiments of the present invention, the benzenesulfonic acid (BA) is comprised at a concentration of about 5 mg/mL to about 30 mg/mL, preferably about 10 mg/mL to about 27.5 mg/mL, more preferably about 10 mg/mL to about 25 mg/mL, most preferably about 10 mg/mL to about 20 mg/mL.

In some embodiments of the present invention, the camphorsulfonic acid may be comprised as camphorsulfonate, which is a pharmaceutically acceptable salt thereof. The camphorsulfonic acid may be comprised as a salt of alkali metal or alkaline earth metal such as sodium, potassium, lithium, calcium, or magnesium, but is not limited thereto.

In some embodiments of the present invention, the camphorsulfonic acid (CA) is comprised at a concentration of about 5 mg/mL to about 30 mg/mL, preferably about 10 mg/mL to about 27.5 mg/mL, more preferably about 10 mg/mL to about 25 mg/mL, most preferably about 10 mg/mL to about 20 mg/mL.

In some embodiments of the present invention, the total concentration of benzenesulfonic acid and camphorsulfonic acid is about 10 mg/mL to about 60 mg/mL, preferably about 20 mg/mL to about 50 mg/mL, more preferably about 20 mg/mL to about 60 mg/mL, about 40 mg/mL, most preferably about 30 mg/mL to 40 mg/mL.

In some embodiments of the present invention, the benzenesulfonic acid and the camphorsulfonic acid may be comprised in a weight ratio of 1:0.5 to 1:2.5, preferably in a weight ratio of 1:1 to 1:2.5, more preferably in a weight ratio of 1:2.0, 1:1 or 1:0.75.

In some embodiments of the present invention, the viscosity-reducing excipient may be used to reduce the viscosity of the high-concentration protein formulation.

In some embodiments of the present invention, the high-concentration protein formulation may be a liquid formulation.

In some embodiments of the present invention, the concentration of protein in the high-concentration protein formulation is about 1 mg/mL or more, about 10 mg/mL or more, or about 50 mg/mL or more, more preferably about 150 mg/mL or more, more preferably about 200 mg/mL or more.

In some embodiments of the present invention, the concentration of the protein is about 10 mg/mL to about 5,000 mg/mL, preferably about 50 mg/mL to about 2,500 mg/mL, more preferably about 100 mg/mL to about 1,000 mg/mL, more preferably about 100 mg/mL to about 500 mg/mL, and most preferably about 150 mg/mL to about 500 mg/mL, but is not limited thereto.

In some embodiments of the present invention, the concentration of the protein is about 1 w/v % to about 70 w/v %, more preferably about 5 w/v % to about 50 w/v %, more preferably about 7 w/v % to about 35 w/v %, more preferably about 10 w/v % to about 25 w/v %, but is not limited thereto.

In one embodiment of the present invention, the protein was comprised at a concentration of about 200 mg/mL (about 20% w/v), but is not limited thereto.

In some embodiments of the present invention, the viscosity-reducing excipient may further contain proline or glycine.

In some embodiments of the present invention, the glycine or proline may be comprised at a concentration of about 10 mM to about 1,000 mM, preferably about 50 mM to about 500 mM, more preferably about 100 mM to about 400 mM, more preferably about 150 mM to about 300 mM. In one embodiment of the present invention, the composition containing 250 mM glycine or proline was prepared, but is not limited thereto.

In another aspect, the present invention is directed to a method for reducing the viscosity of a composition containing a protein, the method comprising adding a benzenesulfonic acid and camphorsulfonic acid as a viscosity-reducing excipient to a composition containing at least one protein, and glycine or proline.

### EXAMPLE

Hereinafter, the present invention will be described in more detail with reference to examples. However, it will be obvious to those skilled in the art that these examples are provided only for illustration of the present invention and should not be construed as limiting the scope of the present invention.

### Example 1. Preparation of High-Protein Concentration Formulations

In order to confirm the viscosity-reducing effect in the high-concentration protein formulation by a combination of benzenesulfonic acid and camphorsulfonic acid, sulfonic acid (BA) and/or camphorsulfonic acid (CA) was added as a viscosity-reducing excipient (Table 2) to a base formulation containing 20% human IgG (Human Immunoglobulin G, Green Cross) and proline or glycine (Table 1) to prepare a liquid composition.

TABLE 1
Base formulation

| Ingredient | Concentration |
|---|---|
| Protein | 20% IgG (w/v) |
| Glycine or Proline | 250 mM |
| Polysorbate 80 | 0.02% (w/V) |

TABLE 2
Viscosity-reducing excipient

| | Viscosity-reducing excipient |
|---|---|
| Formulation | (concentration, mg/mL) |
| Base formulation 1 | None (Table 1) |
| (20% IgG, Proline) | |
| Base formulation 2 | None (Table 1) |
| (20% IgG, Glycine) | |
| BA 10 (Proline) | Benzenesulfonic acid (10 mg/mL) |
| CA 10 (Proline) | Camphorsulfonic acid (10 mg/mL) |
| BA 20 (Proline) | Benzenesulfonic acid (20 mg/mL) |
| CA 20 (Proline) | Camphorsulfonic acid (20 mg/mL) |
| BA 5 + CA 5(Proline) | Benzenesulfonic acid (5 mg/mL); |
| | Camphorsulfonic acid(5 mg/mL) |
| BA 10 + CA 10(Proline) | Benzenesulfonic acid (10 mg/mL); |
| | Camphorsulfonic acid (10 mg/mL) |
| BA 15 + CA 15(Proline) | Benzenesulfonic acid (15 mg/mL); |
| | Camphorsulfonic acid (15 mg/mL) |
| BA 10 + CA 20(Proline) | Benzenesulfonic acid (10 mg/mL); |
| | Camphorsulfonic acid (20 mg/mL) |
| BA 20 + CA 20(Proline) | Benzenesulfonic acid (20 mg/mL); |
| | Camphorsulfonic acid (20 mg/mL) |
| BA 25 + CA 25(Proline) | Benzenesulfonic acid (25 mg/mL); |
| | Camphorsulfonic acid (25 mg/mL) |
| BA 12.5 + CA 27.5(Proline) | Benzenesulfonic acid (12.5 mg/mL); |
| | Camphorsulfonic acid (27.5 mg/mL) |
| BA 30 + CA 30(Proline) | Benzenesulfonic acid (30 mg/mL); |
| | Camphorsulfonic acid (30 mg/mL) |
| BA 20 + CA 15 (Glycine) | Benzenesulfonic acid (20 mg/mL); |
| | Camphorsulfonic acid(15 mg/mL) |

### Example 2. Confirmation of Viscosity-Reducing Effect

of high-concentration protein formulations under low-temperature refrigeration conditions

### Example 2-1: Viscosity Measurement Method

In order to measure the viscosity of the proline-based 20% IgG formulation, the viscosity of 500 μL of each viscosity-reducing formulation sample shown in Table 2 was measured under 4° C. refrigeration conditions using RheoSense's mVROC according to the developer's protocol.

### Example 2-2: Viscosity-Reducing Effect of Proline-Based 20% IgG Formulation

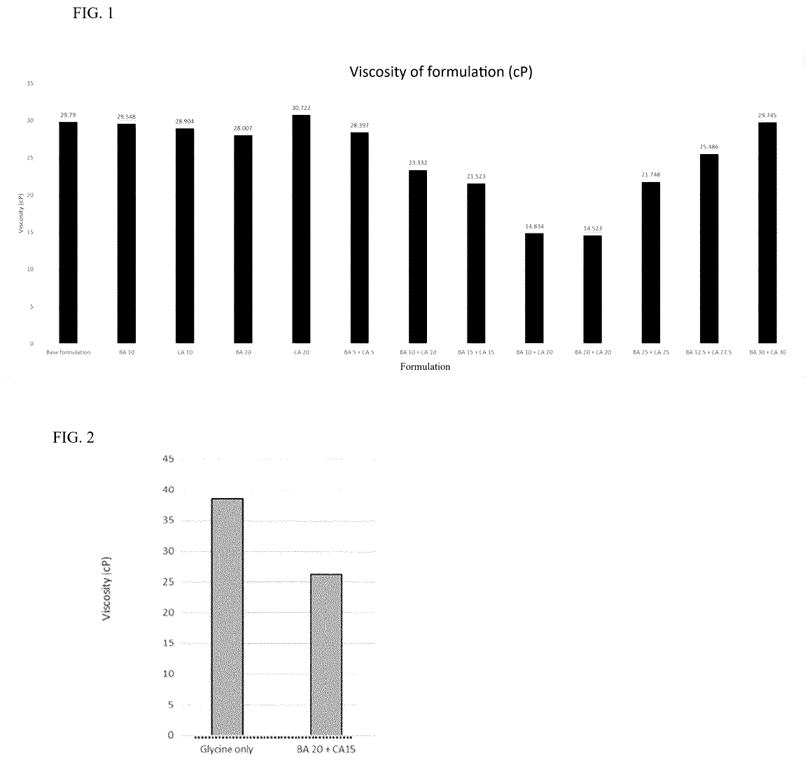

The viscosity of the 20% IgG protein formulation containing proline after refrigeration at 4° C. is shown in Table 3 below (FIG. 1).

TABLE 3
Viscosity of proline-based formulation (4° C.)

| Formulation (Proline) | Viscosity (cP) |
|---|---|
| Base formulation | 29.79 |
| BA 10 (Proline) | 29.548 |
| CA 10 (Proline) | 28.904 |
| BA 20 (Proline) | 28.007 |
| CA 20 (Proline) | 30.722 |
| BA 5 + CA 5 (Proline) | 28.397 |
| BA 10 + CA 10 (Proline) | 23.332 |
| BA 15 + CA 15 (Proline) | 21.523 |
| BA 10 + CA 20 (Proline) | 14.834 |
| BA 20 + CA 20 (Proline) | 14.523 |
| BA 25 + CA 25 (Proline) | 21.748 |
| BA 12.5 + CA 27.5 (Proline) | 25.486 |
| BA 30 + CA 30 (Proline) | 29.745 |

As shown in Table 3, when benzenesulfonic acid or camphorsulfonic acid was added alone, no effect was shown on the viscosity regardless of the concentration thereof. However, when benzenesulfonic acid and camphorsulfonic acid were administered in combination, the viscosity decreased. In particular, each of benzenesulfonic acid and camphorsulfonic acid was added in an amount of 10 mg/mL, 15 mg/mL or 25 mg/mL (BA 10+CA 10, or BA 25+CA 25), or a combination of 12.5 mg/mL of benzenesulfonic acid and 27.5 mg/mL (BA 12.5+CA 27.5) of camphorsulfonic acid was added, viscosity-reducing effect of about 20% to 30% was obtained under low-temperature refrigerated storage conditions, and when a combination of 10 mg/mL of benzenesulfonic acid and 20 mg/mL of camphorsulfonic acid (BA 10+CA 20), or 20 mg/mL (BA 20+CA 20) of each of benzenesulfonic acid and camphorsulfonic acid was added, the viscosity decreased by more than 50%.

On the other hand, when each of benzenesulfonic acid and camphorsulfonic acid was added in an amount of 30 mg/mL or more, the viscosity-reducing effect of the base formulation was not obtained, which means that the viscosity-reducing effect by benzenesulfonic acid and camphorsulfonic acid does not simply increase in proportion to the concentration, but is caused by the specific concentration and concentration ratio defined by the present invention.

### Example 2-3: Viscosity-Reducing Effect of Glycine-Based 20% IgG Formulation

The viscosity of the 20% IgG protein formulation containing glycine after refrigeration at 4° C. is shown in Table 4 below (FIG. 2).

TABLE 4
Viscosity of glycine-based formulation (4° C.)

| Formulation (Glycine) | Viscosity (cP) |
|---|---|
| Base formulation | 38.557 |
| BA 20 + CA 15 (Glycine) | 26.154 |

When a formulation contains glycine, a combination of benzenesulfonic acid and camphorsulfonic acid provides a viscosity-reducing effect of about 33% compared to a formulation not containing the same.

### INDUSTRIAL APPLICABILITY

The excipient composition for reducing the viscosity of protein pharmaceuticals, and the low-viscosity, high-concentration protein formulation containing the same according to the present invention have a remarkably low viscosity even at a protein concentration of about 20% or more, make to manufacture, storage, and use convenient, and are particularly useful for the preparation of injections for antibody treatment for subcutaneous administration and intramuscular administration. In particular, the high-concentration protein formulation of be advantageously used the present invention can immediately after storage without separate preheating because it maintains low viscosity even when stored at low temperature.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

What is claimed is:

1. A pharmaceutical composition comprising:
at least one protein;
glycine or proline;
benzenesulfonic acid; and camphorsulfonic acid.

2. The pharmaceutical composition according to claim 1, wherein the protein is an antibody or an antigen-binding fragment thereof.

3. The pharmaceutical composition according to claim 1, wherein the protein is comprised at a concentration of 10% to 25% (w/v).

4. The pharmaceutical composition according to claim 1, wherein the glycine or protein is comprised at a concentration of 150 mM to 250 mM.

5. The pharmaceutical composition according to claim 1, wherein the benzenesulfonic acid and the camphorsulfonic acid are each independently comprised at a concentration of 10 mg/mL to 27.5 mg/mL.

6. The pharmaceutical composition according to claim 1, wherein a total concentration of the benzenesulfonic acid and the camphorsulfonic acid is 20 mg/mL to 50 mg/mL.

7. The pharmaceutical composition according to claim 1, wherein the benzenesulfonic acid and the camphorsulfonic acid are comprised in a weight ratio of 1:0.5 to 1:2.5.

8. The pharmaceutical composition according to claim 1, wherein the pharmaceutical composition has an absolute viscosity of less than 25 cP at 4° C.

9. The pharmaceutical composition according to any one of claims 1 to 8, wherein the pharmaceutical composition is administered by subcutaneous injection.

10. A method of reducing a viscosity of a composition containing protein, the method comprising adding a benzenesulfonic acid and camphorsulfonic acid as a viscosity-reducing excipient to a composition containing at least one protein, and glycine or proline.

11. The method according to claim 9, wherein the protein is an antibody or an antigen-binding fragment thereof.

12. The method according to claim 9, wherein the protein is comprised at a concentration of 10% to 25% (w/v).

13. The method according to claim 9, wherein the glycine or protein is comprised at a concentration of 150 mM to 250 mM.

14. The method according to claim 9, wherein a total concentration of the benzenesulfonic acid and the camphorsulfonic acid is 20 mg/mL to 50 mg/mL.

15. The method according to claim 9, wherein the benzenesulfonic acid and the camphorsulfonic acid are comprised in a weight ratio of 1:0.5 to 1:2.5.
